SEQUENCE LISTING (2 sequences)

SEQ ID NO: 1 - DNA, 18 bp, Artificial
  Description of Artificial Sequence Synthetic Oligonucleotide
atgtccacactggtgata

SEQ ID NO: 2 - RNA, 65 nt, Artificial
  Description of Artificial Sequence Synthetic Oligonucleotide
gggagaccacaacgguuucccucuagaaauaauuuuguuuaacuuuaagaaggagauauaccaug

(19) United States
(12) Patent Application Publication
(10) Pub. No.: US 2006/0024679 A1
(43) Pub. Date: Feb. 2, 2006

(54) Method for predicting the expression efficiency in cell-free expression systems

(75) Inventors: Dieter Voges, Munchen (DE); Bernd Buchberger, Peissenberg (DE); Sabine Wizemann, Bichl (DE); Manfred Watzele, Weilheim (DE); Cordula Nemetz, Streitdorf (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Biomax Informatics AG, Martinsried (DE)

(21) Appl. No.: 10/913,250

(22) Filed: Aug. 6, 2004

Related U.S. Application Data
(63) Continuation of application No. PCT/EP03/01251, filed on Feb. 7, 2003.

(30) Foreign Application Priority Data
Feb. 7, 2002 (DE) 102 05 091.0

(51) Int. Cl. C12Q 1/68; G01N 33/48; G01N 33/50; G06F 19/00

(52) U.S. Cl. 435/6; 702/20

(57) ABSTRACT

The invention relates to a method for the analysis and optimization of the expression efficiency in the preparation of a protein in expression systems and to a method for the preparation of proteins in such expression systems.

### BACKGROUND OF THE INVENTION

Many expression systems are available for the preparation of proteins. A distinction is made between preparation methods which use live cells, such as E. coli, yeast, mammalian cell cultures and insect cells, i.e. are based on cellular or in vivo expression systems, and preparation methods which use subcellular fractions from suitable organisms, such as E. coli lysates, wheat germ lysate or a reticulocyte lysate, i.e. are based on cell-free or in vitro expression systems.

Each of the above methods exhibits advantages and disadvantages. For instance, cellular expression systems are widely used, can easily be scaled up and may allow the preparation of proteins with secondary modifications. The disadvantages include the necessity of having the appropriate infrastructure, frequently laborious optimization steps and the requisite expert knowledge.

Cell-free expression systems have only recently been optimized to such a degree that they can compete with cellular systems with respect to productivity and scaling. The advantages of this approach include simplicity and flexibility in handling, simple labeling of proteins, expression of cell-toxic proteins and the possibility of parallel expression of for example hundreds of proteins. Prokaryotic systems, specifically E. coli, are preferably used, because of the simple growth conditions and the well established molecular biological and genetic methods. As a consequence of activities in the field of genome sequencing there is an increasing demand for simple, robust and efficient methods for protein expression.

A weak point in the preparation of proteins in both cellular and cell-free expression systems is the unreliable predictability of the expression yield. This is especially the case when the gene of one organism, e.g. human, is to be introduced into a heterologous system, e.g. E. coli, for expression. The reasons for this include especially species-specific peculiarities of using the genetic code, particularly signal sequences and the often multifarious regulation mechanisms. This is often a matter of trial and error, so that achieving the desired result requires major expenditure of time and money.

The preparation of peptides and proteins in cellular or cell-free prokaryotic expression systems is described in detail in the state of the art. Such systems employ DNA matrices such as plasmids and often use a bacteriophage RNA polymerase. In the case of cellular expression, the cell provides substrates, cofactors and accessory enzymes.

In contrast, cell-free protein synthesis requires that some of the substrates, such as nucleotides and amino acids, have to be added to the lysate. For example, a kit for in vitro expression contains a specially prepared E. coli extract, a mixture of all necessary substrates and a secondary energy substrate, such as creatine phosphate, phosphoenolpyruvate or acetylphosphate. The coding sequence for the protein to be prepared is present as an expression construct, i.e. it is surrounded at an optimal distance by the regulatory elements necessary for expression. An expression construct of a sequence which is to be expressed in E. coli or in a lysate of E. coli cultures with the help of T7 polymerase thus ideally contains a T7 promoter sequence, a prokaryotic ribosome binding site and a T7 terminator sequence at an optimal distance from each other. In the first step, the protein-coding sequence is cloned into an expression vector suitable for the selected expression system, or a linear expression construct is produced by PCR techniques. The DNA is then transcribed into mRNA in the prokaryotic expression system with the help of the components described. The mRNA is then translated into the protein. In cell-free systems, both transcription and translation take place in the reaction vessel and are coupled. The substrates which are necessary for the maintenance of the reaction can also be added continuously, for example, through a semipermeable membrane. The transcription of DNA into mRNA occurs at about the same efficiency for expression constructs of different genes. The quantity expressed by each protein thus largely depends on the efficiency of translation by the E. coli ribosomes.

The terms host and host organism will hereinafter be used also to describe cell-free expression, and mean the organism which is the source of the cell extract or cell lysate used for in vitro expression.

With the techniques which have been used up to now, it is not possible to predict the quantity of protein which will be produced by an expression system, in particular a prokaryotic expression system. This is partly because not all factors are known which influence the expression and partly because the exact quantitative effects and synergistic effects of known factors cannot be assessed.

Currently known factors influencing the expression efficiency include the type of promoter, the type of protein, the codon usage as well as the secondary structure of the mRNA. There are also indications that translation efficiency is highly dependent on its initiation, particularly on the secondary structure of the initiation region. The initiation region is located before the translation start codon and includes the Shine-Dalgarno region. The first two codons of the gene may also possibly be part of the initiation region.

Another recognized influence is what is known as the codon adaptation index. This shows how well a nucleotide sequence is adapted to the codons of well expressed proteins of the host, from which for instance the translation apparatus for in vitro synthesis originates. The degeneration of the genetic code permits “unfavorable” codons to be replaced with “favorable” codons, thus optimizing the codons for efficient synthesis. The disadvantage of this procedure is the requirement for extensive experimental work, since the gene to be expressed has to be assembled from a multitude of synthetically produced nucleotide fragments.

Although it has now been possible to express a large number of proteins successfully by using prokaryotic expression systems, the current success rate when using these systems is only about 50%. Protein expression can be optimized in many cases by using different expression vectors, by introducing N- or C-terminal sequence tags or by the substitution, deletion or insertion of one or several nucleotides within the coding sequence (Nemetz C., Watzele M., Wizemann S., Buchberger B., Metzler T., Zaiss K., Fernholz E., Mutter W.; Optimization of the translation initial region of prokaryotic expression vectors for high level in vitro-protein synthesis; 18^{th} Int. Congr. of Biochem. and Mol. Biol. 2000). However, a trial and error optimization of this sort can also lead to reduced expression or to the partial or total loss of function. Moreover, the yields of product are very difficult to predict, because, as described above, cell-free transcription and translation are influenced by numerous factors, and especially by the expression construct used.

Therefore, on the basis of the findings described above, it has not yet been possible to predict the expression efficiency on the basis of the coding sequence of the protein to be prepared.

Hence, it would be desirable to have a method for predicting the success rate of protein preparation in expression systems and possibly for suggesting ways of improving the expression efficiency before the experiment has been performed.

### SUMMARY OF THE INVENTION

It is therefore an object of the invention to provide a method for predicting, on the basis of a given coding DNA sequence, the expression efficiency when preparing a protein in an expression system, preferably in a prokaryotic expression system, particularly preferably in a cell-free expression system. A further object is to provide a method with which the selection of the expression construct can be optimized based on the predicted expression efficiency. Further objects can be derived from the following description.

The features of the independent patent claims serve to fulfill these and other objects.

Advantageous embodiments are defined in the respective dependent claims.

It is now possible for the first time to make available a method which allows a highly accurate prediction of the expression efficiency or yield when preparing a protein in expression systems, preferably in prokaryotic expression systems, on the basis of a given nucleotide sequence coding for the protein to be prepared. Moreover, on the basis of the method according to the invention an optimized construct for protein synthesis can be provided. Constraints selected by the user can be considered in this process.

High accuracy in the context of the present invention means accuracy of at least 50%, preferably of at least 60%, more preferably of at least 75%, particularly preferably of at least 85% and most preferably of at least 95%. The accuracy of the prediction is defined as the number of correctly predicted expression yields divided by the number of predicted expression yields. In the context of the present invention, the expression quantity or yield for a sequence is deemed to be correct if the actual, that means experimentally observed expressed quantity of this sequence lies above or below a defined threshold value and the predicted expressed quantity of the same sequence also lies above or below this threshold value.

In addition, a measure of the accuracy of predictability can be given by analyzing the difference between the predicted and actual expression yield. Thus, in the context of the present invention, the expression efficiency or the yield is deemed to have been correctly predicted when the difference between the calculated and actual yields is not more than 0.4 REU, preferably not more than 0.35 REU and particularly preferably not more than 0.3 REU, wherein 1 REU stands for a reference expression unit and corresponds to the quantity expressed of a well expressed protein in the observed translation system. 1 REE is taken here as being the quantity expressed of the green fluorescence protein (GFP).

On the basis of the given coding sequence, initially one or several, preferably at least 50, particularly preferably at least 100 and most preferably at least 1000 expression constructs or expression vectors are generated. In the context of the present invention, the generation or production of an expression construct means the provision of a construct as a possible starting material for protein synthesis, the expression construct corresponding to the mRNA coding for the protein. The generation is then usually not synthetic, but theoretical, as part of synthesis planning.

During the generation, sequence segments having regulatory elements such as a promoter sequence, a ribosome binding site and/or a transcription termination sequence are preferably placed before and after the coding region. These sequence segments should be compatible with the expression system to be used, such as an expression system based on E. coli and T7 RNA polymerase.

In order to increase the likelihood of successful expression, mutations in the coding region and in the regulatory regions can be performed during generation. The mutations can, for example, be selected in such a way that they lead to identical amino acids, conservative amino acid substitutions, amino acid deletions and/or amino acid insertions in the translation product. If mutations are made in the regulatory sequence before the start codon, care must be taken that these elements are functional, depending on the sequence and from the distances to the start codon.

Essential regulatory elements include a transcription promoter, the ribosome binding site and possible translation enhancing sequences. A preferred promoter for prokaryotic expression is, for example, the promoter of phage T7, which ensures a high transcription rate. The T7 promoter sequence has been described by Moffat, B. A. & Studier, F. W. (1986) J. Mol. Biol. 189, 113. For example, an optimal ribosome binding site (Shine, J & Dalgarno, L. (1974) Proc. Natl. Acad. Sci. USA 71, 1342) and the optimal distance between this site and the start ATG (Chen, H. et al (1994) Nucl. Ac. Res. 22, 4953) were shown for the optimal translation rate of the prokaryote E. coli. As an additional translation enhancing element for E. coli, Olins P. O. et al ((1988) Gene 73, 227) have identified a regulatory element region T7 gene 10 which is frequently used in prokaryotic expression constructs. Mutations can also be performed between and, to some extent, within these elements, to obtain optimized expression constructs.

At least one, preferably several, attribute values are then determined for each of these expression constructs. In the context of the present invention, the term attribute values means properties or characteristics of the expression construct which influence the expression efficiency of a protein, particularly in vitro. These factors, which are important for the expression quantity, can be identified, inter alia, using statistical routines. Examples of attribute values include the G/C content of the coding sequence, codon adaptation indices and base pairing probabilities for each codon or base in the sequence.

The expression quantity or yield to be expected is calculated by mutual linkage of these attribute values and possibly a sequence for expression constructs corresponding to the expected expression quantity is drawn up. Mutual linkage of the attribute values is preferably derived from the analysis of experimental expression results, so-called training data. For this purpose, the expression yields are determined experimentally for a large number of expression constructs and the dependence of the yield on specific attribute values is subsequently investigated. For example using regression procedures a mutual relationship for calculating the expression efficiency or yield subject to defined attribute values can be determined in this way (W. W. Cooley, P. R. Lohnes, Multivariate Data Analysis, John Wiley, New York 1971, page 49 ff).

The subject of the present invention is therefore a method for predicting the expression efficiency in the preparation of a protein in expression systems, preferably in prokaryotic expression systems, the method comprising the following steps:
A) Generating at least one expression construct, comprising a sequence coding for the protein and flanking sequences, particularly regulatory sequences;
B) Determining at least one attribute value of the expression construct influencing the expression efficiency; and
C) Calculating the expression efficiency of the expression construct by mutual linkage with at least one attribute value determined in step B).

The invention thus provides a method which allows highly accurate prediction of the expression yield, particularly in prokaryotic expression systems. Using the method according to the invention, by varying or modifying the constructs, an expression construct can be provided which is optimized for the relevant expression system and the protein to be prepared. In this way, the expression efficiency in expression systems, in particular in prokaryotic expression systems, can be considerably improved. Apart from the expression efficiency, other information about the product being prepared can be provided for a given coding sequence, such as electrophoretic mobility, solubility, dependence on chaperones, and the like.

The method according to the invention is therefore suitable for the prediction of expression efficiency for both cellular and cell-free protein expression. Prokaryotic expression systems which can be used thus comprise cellular expression systems based on prokaryotic cells and cell-free expression systems based on extracts from prokaryotic cells. E. coli is particularly preferred as prokaryotic cell or for the preparation of a cell extract.

The attribute values of the coding sequence which determine the expression efficiency are particularly selected from the group consisting of quantitative primary structure attributes, qualitative primary structure attributes and quantitative secondary structure attributes.

In the context of the present invention, quantitative primary structure attributes mean attributes which are determined by the frequency of occurrence of monomer components, such as the bases A, T, G and C in certain regions or in the overall primary structure of the expression construct. A quantitative primary structure attribute is, for example, the G/C content in a subregion or in the whole region of the coding sequence.

In the context of the present invention, qualitative primary structure attributes means attributes which are related to the type of monomer components, such as the bases A, T, G and C, in certain regions or in the overall primary structure of the expression construct. An example of a qualitative primary structure attribute would be the first base of the second codon of the coding sequence and/or the base sequence of the second codon.

In the context of the present invention, quantitative secondary structure attributes mean attributes which are determined by the secondary structure of the expression construct or of the transcribed mRNA sequence. An example of a quantitative secondary structure attribute would be the mRNA base pairing probability for at least one of the bases of the coding sequence and of the sequence preceding it, particularly the sequence in the region 40 bases upstream and 40 bases downstream of the start codon ATG, particularly preferably in the region of 100 bases upstream and 60 bases downstream of the start codon and most preferably within the first bases of the protein coding sequence. The base pairing probability represents the probability of base pairing within the nucleic acid strand of the mRNA, wherein the expression efficiency is lower the more base pairs of this sort are formed. The base pairing probability represents the probability of base pairing within the nucleic acid strand of the mRNA; the more base pairs of this type are formed, the lower will be the expression efficiency.

As already mentioned above, in step A) of the method according to the invention to optimize expression efficiency, mutations can be made in both the coding region as well as in the regulatory region. In a preferred embodiment of the method according to the invention, expression efficiency is thus determined for coding sequences which differ from the native sequence coding for the protein to be prepared by at least one base substitution in the coding sequence. Particularly preferred are base substitutions which lead to identical amino acids or to conservative amino acid substitutions in the protein to be prepared. In the context of the present invention, conservative amino acid substitution means substitutions in which amino acid are substituted by other amino acids with similar functionalities, charges, polarities or hydrophobicities, for example, substitution of a glutamine residue by an asparagine residue. In an alternative embodiment of the method according to the invention, amino acid substitutions are also conceivable in which one amino acid is substituted by any other amino acid. The number or type of the permitted amino acid substitutions are therefore an essential parameter of this preferred embodiment of the expression construct in step A) of the method according to the invention.

The base substitutions preferably occur in the first codons, for example in the first 10 or 20 codons of the coding sequence. It is also preferred that base substitutions are performed in not more than seven codons of the coding sequence. Moreover, it is preferred that the G/C content should not exceed 0.7 in each codon in which one or more base substitutions are performed. An additional preferred feature when introducing mutations into the coding region is that the codon adaptation index should be at least 0.02 for each codon in which one or more base substitutions have taken place. The codon adaptation index corresponds to the use of a codon in a specific gene relative to the overall use of this codon in expressed genes in the same host. The codon adaptation index indicates the use of codons in a certain gene relative to the overall use of this codon in expressed genes of a host organism.

The calculation of expression efficiency for each of the expression constructs by mutual linkage with the determined attribute values is preferably performed by multiple regression, for example, by linear regression of the dependence of experimentally determined expression yields on attribute values of the corresponding expression constructs. The suitability of regression analysis for the method according to the invention increases with the quantity of experimental data available for its determination, in other words, with the number of expression constructs for which the dependence of the expression yield on specific attribute values has been experimentally determined. As attribute values or independent variables used in the regression, the G/C content and/or the base pairing probability are mainly used.

In a further preferred embodiment, expression efficiency for each of the expression constructs is calculated by mutual linkage with the attribute values determined in step B) by using computer-learning methods which construct a decision tree out of a set of cases belonging to known classes (J. R. Quinlan, C4.5: Programs for Machine Learning, Morgan Kaufmann, San Mateo, Calif., USA 1993).

In a further preferred embodiment, expression efficiency for each of the expression constructs is calculated by mutual linkage with the attribute values determined in step B) by using neural networks (see e.g. D. E. Rumelhart et al, “Learning Representations by Back-Propagating Errors”, Nature 1986, 323, 533-636; R. Hecht-Nielsen, “Theory of the Backpropagation Neural Network”, in Neural Networks for Perception, pp. 65-93 (1992); D. Nauck, F. Klawonn, R. Kruse, Neuronale Netze und Fuzzy-Systeme (Neural Networks and Fuzzy Systems), Vieweg-Verlag, 1994; Rüdiger Brause, Neuronale Netze (Neural Networks), Teubner-Verlag, 1995; R. Rojas, Theorie der Neuronalen Netze (Theory of Neuronal Networks), Springer-Verlag, 1993; A. Zell, Simulation Neuronaler Netze (Simulation of Neural Networks); Addison-Wesley, 1994).

In a further preferred embodiment, the calculation of expression efficiency for each of the expression constructs is performed with a Bayes network (J. Pearl, Probabilistic Reasoning in Intelligent Systems, Morgan Kaufmann, San Mateo, Calif., USA, 1988).

In a further preferred embodiment, the expression constructs and their protein products are analyzed for undesired fragmentation sites. Corresponding expression constructs are then generated in step A) in which this fragmentation is minimized. Examples of undesired fragmentation sites within the coding sequence include internal initiation sites, sites of premature termination and/or rare codon clusters. Undesired fragmentation sites within the protein product are particularly proteolytic cleavage sites.

In a specific embodiment using expression vectors, the method according to the invention includes the following steps:
a) Providing a nucleic acid sequence which codes for the protein to be prepared;
b) Specifying constraints for the desired cloning strategy, the incorporation of purification and/or detection tags and/or the number or type of permitted amino acid substitutions;
c) Selecting a suitable expression vector;
d) Generating at least one expression construct containing the native sequence coding for the protein;
e) Generating one or more modified expression constructs by nucleotide substitutions and/or insertions and/or deletions;
f) Calculating the expression efficiency for each of the expression constructs by mutual linkage with at least one of the attribute values influencing the expression efficiency;
g) Outputting the expression efficiency calculated for the expression construct(s)

In a further specific embodiment using PCR-generated matrixes, the method according to the invention includes the following steps:
a) Providing a nucleic acid sequence coding for the protein to be prepared;
b) Specifying constraints for the incorporation of purification and/or detection tags and/or the number or type of permitted amino acid substitutions;
c) Generating at least one expression construct containing the native sequence coding for the protein;
d) Generating one or more modified expression constructs by nucleotide substitutions and/or insertions and/or deletions;
e) Calculating the expression coefficient for each of the expression constructs by mutual linkage with at least one of the attribute values influencing the expression efficiency;
f) Generating PCR primer sequences;
g) Outputting the expression efficiencies calculated for the expression construct(s) and/or the PCR primer sequences for the expression constructs.

The generation of PCR primer sequences in step f) is generally performed having regard to the rules on PCR primer design, with which the expert is familiar (Newton & Graham, PCR, Spektrumverlag, Heidelberg, Deutschland, 1994; McPherson & Moller, PCR, BIOS Scientific Publishers, Oxford, Great Britain, 2000; Kain et al., 1991)

The above mentioned steps for the embodiments using expression vectors or PCR-generated matrixes, a) to g) in both cases, may be performed singly or in any combination or sequence. When faced with a specific problem, the expert is able to decide on a suitable selection and sequence of the method steps given above.

In addition, the method according to the invention can include the provision of data on the physicochemical properties of the protein product and of suggestions for their improvement and/or instructions to the individual steps for the preparation of the expression constructs.

The above described method according to the invention for predicting expression efficiency is preferably computerized, in other words, at least one and especially preferably all steps of the method are performed on a computer, for example, a PC. In a specific embodiment of a computerized method of this sort, the coding nucleic acid sequence is provided, for example, in text format.

A further aspect of the present invention relates to a machine-readable medium on which are stored instructions for the performance of the above described method according to the invention, which instructions can be carried out on a computer.

A further aspect of the present invention relates to a computer program product designed so that the above described method according to the invention is effected when the computer program product is used on a computer.

A further aspect of the present invention relates to a method for preparing a protein in cellular expression systems, preferably prokaryotic systems, which comprises the following steps:
a) Predicting the expression efficiency for expression constructs according to the method described above;
b) Selecting an expression construct with a determined expression efficiency, preferably the highest expression efficiency;
c) Cellular expression of the protein based on the expression construct from step b).

A further aspect of the present invention relates to a method for the preparation of a protein in cell-free expression systems, preferably prokaryotic systems, comprising the following steps:
a) Predicting the expression efficiency of the expression constructs according to the procedure described above;
b) Selecting an expression construct with a determined expression efficiency, preferably the highest value;
c) in vitro synthesis of the protein based on the expression construct in step b).

Specific embodiments of the method according to the invention for predicting the expression efficiency of expression constructs will be described below.

### BRIEF DESCRIPTION OF THE DRAWINGS

FIGS. 1a-1c: Generation of circular and linear expression constructs;

FIG. 2: Histogram of the expression values of 742 sequences which solely vary in the 39 bases starting from the second codon—expressed as percentage of the expression of GFP;

FIG. 3: Three dimensional scatter plot of the expression quantity against the mean G/C content and the base pairing probability of the mRNA secondary structure

FIG. 4: a) Initiation region of all sequences shown in the example. esp-g10: Initiation Enhancer Region; SD: Shine-Dalgarno Sequence; ATG: Translation Start Codon; b) Primer Design for the Expression PCR, with the external primers 5′-=C, containing T7 Promoter, Gene 10, RBS, and 3′-=D, containing a non-translated spacer and a T7 transcription terminator; and the internal primers 5′=A, containing RBS, ATG and a gene-specific sequence, and 3′-=B, containing a gene-specific sequence and a spacer region.

FIG. 5: Histogram of the expression values of the sequences from E. coli.

FIG. 6: Histogram of the expression values of the sequences from A. thaliana.

FIG. 7: Histogram of the expression values of the human sequences.

FIG. 8: Histogram of the expression values of the sequences from S. cerevisiae.

FIG. 9: Histogram of the expression values of the viral sequences.

FIG. 10: Illustration of the standard deviations o the binding probabilities ppX.

FIGS. 11-14: Illustration of the correlation values at different temperatures for the base pairing probabilities of different bases with the expression quantity

FIG. 15: Box plot of the mean ppav against the expression categories “none”, “low”, “good” and “high”.

FIG. 16: Histograms of the differences between the predicted and actual expression values

FIG. 17: Illustration of a decision tree to predict the probability of expression.

### DETAILED DESCRIPTION OF THE INVENTION

The generation of one, preferably several, expression constructs for a given nucleic acid sequence comprises a series of steps. The given nucleic acid sequence includes at least the region which codes the protein to be expressed in the prokaryotic expression system. The sequence may contain start and stop codons, depending on the type of sequence, for example, whether it is a sequence which codes for a complete protein or only for a single domain. The given sequence can be analyzed for errors, such as ambiguities or false signs or for the presence of start and stop codons.

As already mentioned, constraints and preferences for expression constructs can be specified in step a) of the method according to the invention in the generation of expression constructs. Constraints include the planned cloning strategy, specific characteristics of the expression constructs, for example, the position and type of a tag and the permissibility of amino acid substitutions.

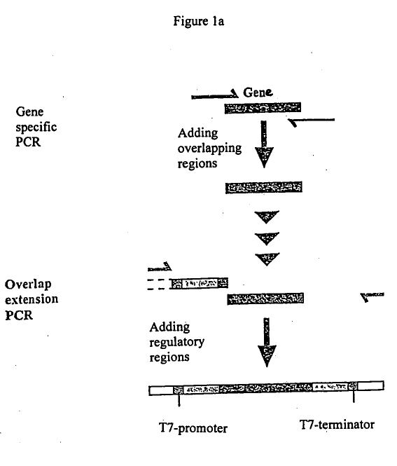

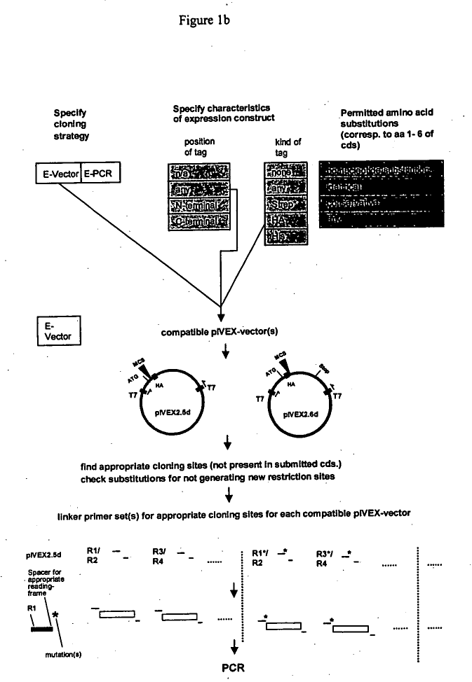

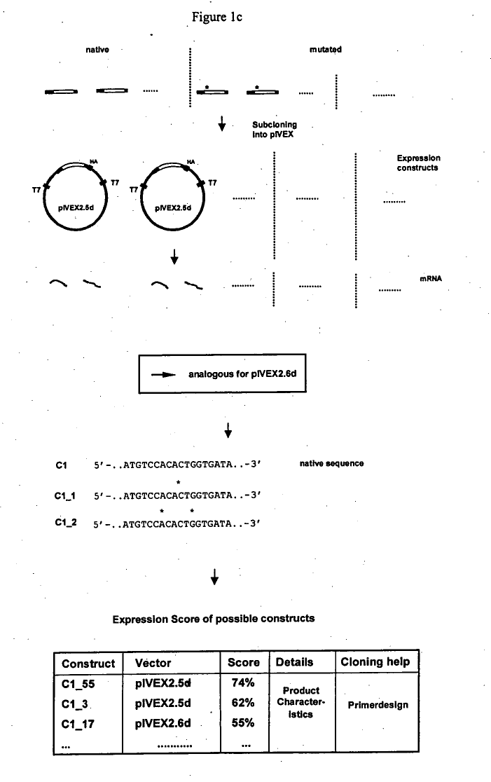

The expression construct can be specified and generated as either a circular or a linear construct (see FIG. 1). If circular expression constructs are used, the analysis includes the identification of suitable restriction sites for cloning. For this reason, the selected vector, e.g. pIVEX (Roche Diagnostics GmbH, Mannheim, Germany), is checked for restriction sites, either at the multiple cloning site or at other sites which are not contained in the given coding sequence (see FIG. 1b). Further, linker primers containing spacer sequences can be developed, to ensure translation in the desired reading frame.

Further, for generating expression constructs in step A) of the method according to the invention, also various kinds of tags can be selected, if these are desired for the synthesis. In the context of the present invention, suitable tags include inter alia those which are conventionally used for the purification or detection of the expression product. Examples of these include Streptag, hexa-histidine-tag or HA tag (see Table 1). Further, the location of the tag can be selected, for example, C- or N-terminal. Alternatively, various fusion constructs, such as maltose binding protein (MBP) and glutathione S transferase (GST), can be selected for the generation of expression constructs.

TABLE 1
Expression cloning vectors pIVEX, which are compatible
with the cell-free E. coli expression system RTS Expert
(Roche Diagnostics) and their characteristics

| | | Factor Xa Restriction | | |
|---|---|---|---|---|
| Cloning | Cloning | Protease cleavage site | Kind of | Position of |
| Vector | Sites | for the Tag | Tag | the Tag |
| pIVEX 2.3d | MCS | — | His | C-terminal |
| pIVEX 2.4d | MCS | X | His | N-terminal |
| pIVEX MBP | MCS | X | His + MBP | N-terminal |
| pF/EX 2.5d | MCS | — | HA | C-terminal |
| pIVEX 2.1d | MCS | — | Streptag | C-terminal |
| pIVEX 2.2d | MCS | — | Streptag | N-terminal |
| pIVEX-GST | MCS | — | His + GST | N-terminal |
| pIVEX 2.6d | MCS | X | HA | N-terminal |

As already described above, alternative nucleotide sequences can be generated to improve the expression efficiency of the resulting constructs. The alternative sequences are preferably also examined for the presence of restriction sites within the coding sequence.

Vector-based expression first requires the selection of that vector which fulfils the given constraints. It is moreover advantageous to generate all expression constructs resulting from a combination of the potential cloning sites in combination with the fixed constraints.

Linear expression constructs can be prepared by various PCR techniques. The regulatory elements necessary for expression are fused on, either with a long external primer or by adding DNA and overlap-extension PCR (Newton & Graham, 1994, see above; McPherson and Moller, 2000, see above; Kain et al., 1991, see above). The latter method has been implemented in the RTS Linear Template Generation Set from Roche Diagnostics. In the first PCR step in this method, overlapping sequences are fused onto the protein coding sequence. In the second PCR step, the regulatory regions are inserted through the overlapping regions by overlap extension (see FIG. 1a). Analogously to the circular expression constructs, tag sequences or fusion protein sequences can be inserted.

For the generation of linear expression constructs, mutations in the coding region of the gene-specific first primer are preferably performed in step A) of the method according to the invention. In addition, mutations can also be inserted in the regulatory region.

Irrespective of the selected strategy for the preparation of expression constructs, the method according to the invention makes it possible to give detailed characteristics of the mRNA and the translation product for each expression construct.

In particular, the computerized carrying out of the method according to the invention permits the provision of a list of expression constructs with the individual calculated expression yield, linkages to the extended mRNA and protein characteristics and/or specific cloning aids. The expression constructs are preferably arranged according to the expected quantities expressed. Constructs derived from mutated coding sequences are only shown if a greater expression yield is expected with them than with the native sequence.

In a further preferred embodiment of the present invention, mutations can be generated in step A) of the method according to the invention. For creating the mutations or deletions and/or insertions, it is particularly suitable to use the initial section of a translated region, as the quantity expressed is strongly influenced by the sequence in this region. Thus, for improved expression mutations are preferably generated in the first codons of the translated region, especially preferred in the first 60 nucleotides; more preferably in the first 30 nucleotides and most preferred in the first 21 nucleotides, including the start codon. The mutations can of course also affect regulatory sequences upstream of the start codon.

The mutations are in particular generated according to the following mutation generating rules, which favor mutations having a positive effect on the predicted quantity expressed. Thus, the number of codons of the coding sequence after the start codon which are changed by mutations is preferably not more than ten, particularly preferably not more than eight and most preferably not more than six. In addition, the codon adaptation index for each codon is preferably at least 0.02, particularly preferably at least 0.05 and most preferably at least 0.07. The G/C content for each codon is preferably not greater than 0.7 and particularly preferably not greater than 0.4.

Further, when generating the expression constructs in step A) of the method according to the invention, it can also be decided which type of amino acid substitution is permitted as a result of the nucleic acid substitution, there being the following three possibilities: nucleotide substitutions which lead to identical amino acids; nucleotide substitutions which lead to conservative amino acid substitution as well as nucleotide substitution which lead to arbitrary amino acids.

Depending on the above-mentioned parameters in connection with the selection of the expression construct and the creation of mutations, a large number of expression constructs can thus be provided for a single given coding sequence, for which expression constructs the expression quantities can be predicted using the method according to the invention.

In step B) of the method according to the invention for predicting expression efficiency, the attribute values of the coding sequence which influence the expression efficiency are determined. Such attribute values of the coding sequence can be selected from quantitative primary structure attributes, qualitative primary structure attributes and/or quantitative secondary structure attributes. Particularly preferred attributes, according to which the given DNA sequence or the expression construct including the coding sequence are analyzed, are the G/C content of sub-regions or the entire region of the coding sequence, the first base of the second codon of the coding sequence, the base sequence of the second codon and/or the base pairing probabilities for at least one of the bases of the coding sequence, preferably within the first 60 bases, particularly preferably within the first 21 bases.

In step C) of the method according to the invention, the expression efficiency of each of the expression constructs is finally calculated, by linkage of or by correlating the attribute values determined in step B). On the basis of the calculated expression efficiency or expression amount, the most promising expression construct can be selected. For correlating the attribute values a prediction-linkage is necessary for each given coding sequence.

Such prediction-linkage is preferably derived from the dependence of experimentally determined expression yields (also referred to below as training data) on the attribute values of the expression constructs used. For this purpose, expression yields from preferably at least 100 sequences particularly preferably at least 500 sequences and most preferably at least 1000 sequences are experimentally determined. In further embodiments, the expression yields are determined experimentally from at least 20, at least 50, at least 250, at least 750 or at least 900 sequences. The predictive linkage or the derived predictive algorithm, based in this way on a collection of training data, can then be used for the calculation of the expression efficiency of the expression constructs generated in step A) of the method according to the invention. The prediction-linkage or the prediction algorithm derived in this way and based on a batch of training data can be used to calculate the expression efficiency for expression constructs generated in step A) of the method according to the invention.

In a particularly preferred embodiment, the predictive linkage is derived from the dependence of the experimentally determined expression yields in a specific system on the attribute values of the expression constructs used. The predictive linkage is then used to calculate the expression efficiency of those expression constructs generated in step A) of the method according to the invention which are compatible with the expression system.

In another particularly preferred embodiment, when compiling the training data the non-translated region lying 5′ to the start codon (5′-UTR, 5′ untranslated region) is left unchanged. Thus, in this embodiment, the predictive linkage is derived from the dependence of experimentally determined expression yields on attribute values of the corresponding expression constructs, these expression constructs only exhibiting differences starting with the second codon of the coding sequence, i.e. in the translated region. Those sequences are preferably analyzed which exhibit differences in the 150 bases; particularly preferred the first 90 bases and most preferred in the first 39 bases starting with the second codon of the coding sequences. However, sequences can also be analyzed which exhibit differences in the 120 bases, the 60 bases or the 30 bases starting with the second codon of the coding sequence.

On the basis of the above described derivation of predictive linkage from experimentally determined data, the translation efficiency of mRNA sequences can be predicted with a high degree of accuracy.

The data analysis of training data will be described in detail by way of an example.

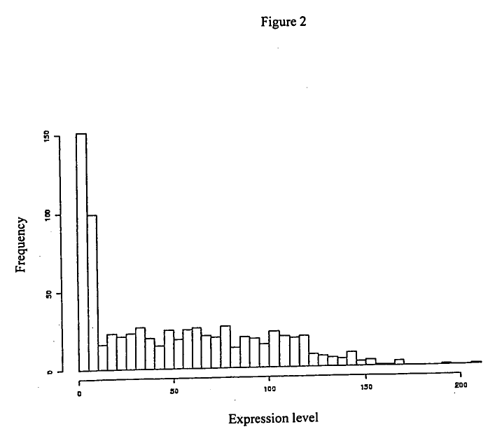

An overview of the essential points of the provision of the predictive linkage from training data will be given below. Thus, for example, the experimental expression data from 742 sequences containing only differences in the 39 bases starting with the second codon of the coding sequence were analyzed for the provision of a data base. FIG. 2 shows a histogram of the expression quantities obtained from in vitro expression of these sequences. The expression quantity is given as a percentage of the expression of the GFP protein fused with the above coding sequences. A broad distribution of expression values is observed, even though the variable region is relatively short.

A series of attribute values was evaluated for each of these sequences. The attribute values which influence the expression quantity can, for example, be identified by correlation analysis or histograms. The G/C content and the base pairing probability within the first 20 to 40 bases of the coding sequence have proven to be the most important sequence attributes. This applies in particular to the specific set of training data examined here, which exhibits no variability in the translation initiation region. The sequence of the initiation region has in general a major influence on the quantity of expressed protein.

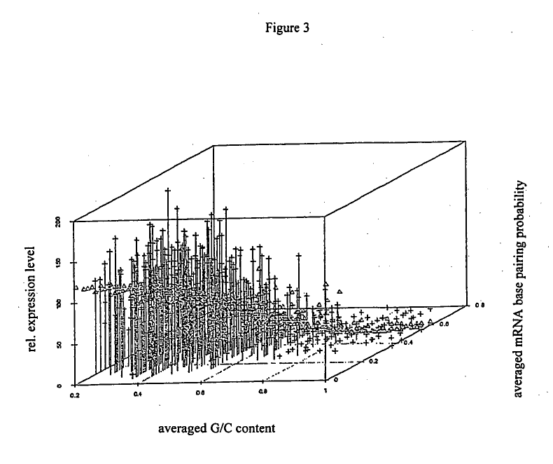

Conventional regression analysis is preferably used for production of the predictive linkage. Thus, for example, the GC content and the base pairing probability—both averaged over a region downstream of the translation codon—may both be used as independent variables for fitting the observed expression levels (see FIG. 3).

Alternatively, a decision tree can be derived with the help of machine-learning methods and this also permits the expression quantity to be predicted.

In order to determine the reliability of the prediction of the mutual linkage used in the method according to the invention with the attribute values determined in step B), the predicted and experimentally measured expression values for a given set of data can be compared. The difference between expected and observed values is plotted in a histogram for the set of data from which the regression model was derived.

Alternatively, only a part of the total data set is used as training data set. The differences between the predicted and experimentally measured quantity expressed is illustrated in a histogram for the rest of the data set, which was not used as training data set.

Another way to evaluate the accuracy of the predictions is to determine the number of correct positives (i.e. positive expression and positively predicted expression), false positives (i.e. negative expression and positively predicted expression), correct negatives (i.e. negative expression and negatively predicted expression) and false negatives (i.e. positive expression and negatively predicted expression). In this context, positive or negative expression means that the quantity expressed was above or below a defined threshold value, preferably given in reference expression units. For example, the threshold value can be a relative expression quantity of 0.30 REE. The accuracy of the prediction is determined from the sum of correct positives and correct negatives, i.e. all correctly predicted cases, divided by the sum of all predicted cases.

The procedure described above for the production of a predictive linkage is based on a data set in which the sequences only vary in the first 39 nucleotides downstream from the translation codon. This region has been identified in the context of the present invention as being particularly essential for the translation efficiency. Training data can of course also be used in which the sequences vary in a larger region, for example, in a region of 40 bases both upstream and downstream from the translation start codon. Moreover, larger regions, for example, within the first 50, 75 or 100 or more nucleotides upstream and/or downstream from the translation start codon can also be varied.

Moreover, training data can also be used in which additional attributes are determined which are present in the regions kept constant in the data set described above. The suitability of a data bank of training data of this sort for the production of a precise predictive linkage increases with the variability of the different attributes, such as the length of the varied sequence, the length of the coding sequence, the length of the mRNA sequence, the codon adaptation indices and the like.

In a further embodiment of the method according to the invention, an additional procedural step is performed in which the given nucleotide sequence and the derived amino acid sequence are examined for critical sites leading to product fragmentation. In addition, the biochemical and functional properties of the product can be characterized. Depending on the results, alternatives can be provided for improving the translation results.

In vitro expression frequently leads to undesired fragmentation of certain proteins. Such fragmentation may be due to differences in the sequence-specific patterns of the mRNA or protein, leading to either incomplete translation or to proteolytic degradation. In this embodiment of the method according to the invention, sequences of this sort can be identified, and preferably at the same time suggestions for minimizing such fragmentation and for increasing in the yield of full-length product can be provided.

For example, product fragmentation can occur when internal translation start sites are present in the coding sequence. Examples of this are Shine-Dalgamo type sequences in proximity to potential initiation codons, which can be recognized by E. coli ribosomes as alternative translation initiation sites. The E. coli initiation codons are AUG (91%), GUG (8%) or UUG (1%) (Makrides, 1996). Such critical sites are found more frequently in eukaryotic genes, because Shine-Dalgarno sites are not necessary for eukaryotic translation and are therefore not eliminated by evolution.

Critical sequence constellations are of special significance for expression yield when the actual start codon is poorly accessible, for example, when the AUG start codon is in a region which forms stable mRNA secondary structure.

Thus, in a preferred embodiment of the method according to the invention the given coding sequence is analyzed for patterns of this sort which can cause fragmentation during expression. If a sequence pattern of this sort is found, recommendations are made for improving the sequence and these can be taken into account in the generation of the expression construct in step A) of the method according to the invention.

In addition, it is known that stable internal 13-structures in mRNA can lead to incomplete translation (de Smit M H, van Duin J.; J. Mol. Biol. 1994, 235, 173-184). As a consequence, based on rules for the prediction of such structures on the basis of the given sequence a corresponding linkage for determining such structures can be developed and can also be taken into account when generating expression constructs.

Another factor which strongly influences gene expression and which is preferably considered in the generation of expression constructs in step A) of the method according to the invention in step A), is the selective use of codons in specific hosts. In general, genes which are rarely expressed contain many more rare codons than highly expressed genes. The use of codons in a specific gene relative to the general use of these codons in the genes expressed in a host is referred to as the codon adaptation index (CAI, Sharp and Li, Nucleic Acids Res. 1987, 15, 1281-1295). Since the codon adaptation index is species-specific, the use of codons for the gene to be expressed should, if at all possible, correspond to that of the host. In a cell-free system, the use of the codons in the organism is considered, from which organism the cell extract used in the in vitro expression has been derived.

In other words, in order to guarantee maximal gene expression, the codon adaptation index should be as high as possible, preferably at least 0.05. For example, the expression yields of mammalian genes in E. coli may be low, because these genes frequently contain the arginine codons AGG and AGA and there are only low levels of the corresponding tRNAs in E. coli. It has often been found that expression increases significantly when rare codons are replaced by codons which are frequently used in an organism (see Makrides, 1996).

The correlation of rare codons with low expression yield is particularly significant when the codons are at the start of the 5′-region of the mRNA, particularly within the first 25 codons of the gene. Local clusters of rare codons can also lead to frame shifts and, in some cases, to premature termination due to abnormal translation pausing.

Thus, in another preferred embodiment of the method according to the invention, an analysis of rare codons in the sequence of the protein to be prepared is performed in step A) of the generation of an expression construct—particularly the first 25 codons—and the difference between the codon usage in the sequence to be expressed and the desired codon adaptation index is determined. On the basis of possible differences, suggestions can be made for conservative base substitutions, to adapt the codons to codons which are frequently used by the host. For example, non-conservative base substitutions are also conceivable, leading to codons which are frequently used in the host and which yield conservative amino acid substitutions. These conservative or non-conservative base substitutions are preferably taken into account in the generation of the expression construct in step A) of the method according to the invention.

Alternatively, it may be suggested that the appropriate tRNAs for the rare codons will be added to the expression reaction.

A further embodiment of the method according to the invention relates to critical cleavage sites in the protein to be prepared. The occurrence of proteolytic degradation caused by proteolytic enzymes in the cell-free lysate, for example from E. coli, can lead to fragmented protein products and is a serious problem in attempts to increase the quantity of full-length protein product expressed. The translated polypeptides can contain amino acids in particular at the N-terminus which are recognized as proteolytic cleavage sites.

Hence, in another preferred embodiment of the method according to the invention, the translated sequence of the protein coded for by the given DNA sequence is therefore analyzed for cleavage sites of this sort, the corresponding proteases of the host preferably being considered. For example, for E. coli proteases an almost complete list can be produced from the data bank SWISS-PROT (see Table 2). The expert is familiar with the specificities from the scientific literature.

If it is not possible to characterize the type of cleavage, the protease is not taken into account, even when it was demonstrated that it caused the degradation of proteins expressed heterologously, e.g. the ion protease.

Some of the proteases shown in Table 2 may have different specificities. The favored cleavage sites (Pn=N-terminal, Pn′=C-terminal) are given in accordance with the Schechter-Berger convention (I. Schlechter and A. Berger, Biochem. Biophys. Res. Commun. 1967, 2:157-162). Amino acids are shown with the one letter code; Xaa stands for an arbitrary amino acid.

TABLE 2
Cytoplasmic proteases, which are potentially contained in an E. coli lysate.

| Enzyme | Type | P1 | P2 | P1′ | P2′ |
|---|---|---|---|---|---|
| Membrane alanyl | Aminopeptidase | A | — | Xaa | Xaa |
| aminotransferase pepN | | | | | |
| [EC 3.4.11.2] | | | | | |
| Membrane alanyl | (Dipeptidylpeptidase) | P | A, V, L, | Xaa | Xaa |
| aminotransferase pepN | | | I, P, W, | | |
| [EC 3.4.11.2] | | | F, M | | |
| Prolyl aminopeptidase | Aminopeptidase | P | — | Xaa | Xaa |
| [EC 3.4.11.5] | | | | | |
| X-Pro-Aminopeptidase APP-II | Aminopeptidase | Xaa | — | P | Xaa |
| [EC 3.4.11.9] | | | | | |
| Bacterial leucyl | Aminopeptidase | L | — | Xaa | Xaa |
| aminopeptidase [EC 3.4.11.10] | | | | | |
| Methionyl aminopeptidase | Aminopeptidase | M | — | Xaa | Xaa |
| MAP [EC 3.4.11.18] | | | | | |
| Alanine carboxypeptidase | Carboxypeptidase | Xaa | Xaa | A | — |
| [EC 3.4.17.6] | | | | | |
| β-Aspartylpeptidase | Aminopeptidase | D | — | Xaa | Xaa |
| [EC 3.4.19.5] | | | | | |
| Omptin ompT [EC 3.4.21.87] | Endopeptidase | K | Xaa | R | Xaa |
| | (Serine) | | | | |
| Pitrilysin Pi [EC 3.4.24.55] | Endopeptidase | Y | Xaa | L | Xaa |
| | [Metallo] | | | | |
| Pitrilysin Pi [EC 3.4.24.55] | Endopeptidase | F | Xaa | Y | Xaa |
| | (Metallo) | | | | |

The classification of the selected enzymes is based on the NC-IOBMB peptidase nomenclature (http://www.chem.qmw.ac.uk/iubmb/enzyme/ec34/).

In a preferred embodiment of the method according to the invention, the positions of the proteolytic recognition sites are identified in the resulting amino acid sequence and the corresponding fragment sizes are calculated for a nucleotide sequence to be expressed in the cell-free expression system.

In addition, information is preferably provided about specific inhibitors and metallic cofactors, substrate specificity, specific activity, optimal temperature and pH values, KN value, stability, etc., of the possible proteases. For example, protease-specific inhibitors can be compared with inhibitors of broad specificity, thus avoiding undesired side-reactions. The use of inhibitors with broad specificity can also inactivate other enzymes, such as methionyl aminopeptidases, which are essential for the removal of the start methionine in about half of all bacterial proteins.

In addition, in another embodiment and on the basis of the analysis of possible cleavage sites, recommendations are made for base substitutions which give conservative or other amino acid substitutions and which lead to avoidance of proteolytic recognition sites in the protein to be prepared. Base substitutions of this sort are preferably considered in the generation of the expression construct in step A) of the method according to the invention.

To attain a high yield of the protein product, if possible with full maintenance of all its functions, it is particularly important to know the characteristics of the protein, particularly its physicochemical properties. Thus, in a particularly preferred embodiment of the method according to the invention and based on the coding nucleotide sequence or the amino acid sequence of the protein, general information on the length, molecular weight, isoelectric point and the like and detailed information on the expected solubility and chaperone dependence is provided. The above-mentioned and additional protein characteristics can be considered in the planning of in vitro protein synthesis.

The yield of fully functional proteins frequently depends on solubility and, for some proteins, also on chaperones. Chaperones are proteins which mediate correct assembly of a target protein by directing its folding to the functionally active conformation. The expression of recombinant eukaryotic proteins, for example, in an E. coli expression system frequently leads to an accumulation of insoluble protein aggregates or “inclusion bodies”. Renaturation of the biologically active products from these aggregated conformations is impossible for many polypeptides, such as structurally complex oligomeric proteins and proteins which contain multiple disulfide bonds.

Protein solubility, or the ability of a protein to form aggregates, is greatly affected by its hydrophobicity and proper folding. Thus, solubility is reduced by clusters of hydrophobic amino acids within a polypeptide, for example, transmembrane domains or signal peptides. In vivo chaperone systems, such as GroEL/GroES in E. coli, catalyze the complex folding of hydrophobic residues from the aggregation-prone protein surface to inner regions. In this way, chaperones avoid improper self-assembly, which occurs in vivo in many proteins as a result of inter- or intramolecular interactions due to hydrophobic sites. In addition, the solubility of a properly folded protein is markedly increased compared to the not properly folded conformation.

In one embodiment of the method according to the invention, the protein sequences to be expressed are analyzed on the basis of the amino acid sequence of the protein coded by the given DNA sequence for clusters of hydrophobic amino acids and transmembrane domains and its solubility predicted in this way. For example, the ALOM2 algorithm can be used for the prediction of transmembrane domains (P. Klein et al., Biochem. Biophys. Acta 1984, 787: 221-226). Conclusions can be drawn from these results about the possible localization of the protein product, e.g. whether it is cytosolic, membrane spanning, etc. If the protein to be expressed turns out to be relatively insoluble, appropriate suggestions can be made for planning its in vitro synthesis, e.g. the addition of mild detergents, lowering the reaction temperature or adding chaperones.

Although proteins can spontaneously fold into their native structures, chaperones can improve the efficiency of the folding by avoiding aggregation and misfolding. The GroEL/GroES-System from E. coli is a particularly preferred chaperone system and is active under all growth conditions. It has been shown in cell-free translation systems that the addition of GroEL/GroES facilitates the native folding of nascent aspartate aminotransferase (J. R. Matingly et al., Arch. Biochem. Biophys. 2000, 382:113-122). More recent studies have shown that about 300 newly translated proteins of different function interact strongly with GroEL and that the maintenance of the conformation is directly dependent on the chaperone for about a third of the proteins (W. A. Houry et al., Nature 1999, 402:147-54).

GroEL substrates preferably contain at least two α/β domains including buried β sheets with large hydrophobic surfaces, and preferably are of molecular weight Mᵣ=20-60 K.

In this embodiment of the method according to the invention, the properties of GroEL substrates described above can be used as a basis for the prediction of GroEL-dependent folding of a protein expressed in vitro, by analyzing the secondary structure of the protein, in particular the SCOP and CATH domains and the molecular weight of the protein.

The DnaK/DnaJ/GRPL chaperone is an additional well studied chaperone system and the protein sequence to be expressed can be analyzed with respect to this. DnaK belongs to the HSP70 protein family. The DnaK system is the major chaperone which prevents the aggregation of a majority of thermolabile proteins. The binding sites and a consensus recognition motive have been derived from the analysis of 37 biologically relevant prokaryotic and eukaryotic proteins which are natural substrates of DnaK or HSP70 (S. Rüdiger et al., EMBO J. 1997, 16:1501-1507). The general features of the substrate-binding sites in HSP70 proteins are conserved within the HSP70 family and thus also in DnaK. The consensus motive recognized by DnaK consists of a central hydrophobic core of four to five residues, particularly leucine, isoleucine, valine, phenylalanine and tyrosine, together with two flanking regions, which are rich in basic residues.

Proteins of the secretory pathway are transported in the periplasma or endoplasmatic reticulum and are labeled by signal sequences. It is estimated that about 10% of all proteins in human and Arabidopsis cells are secretory proteins. The signal sequences are usually located at the N-terminus in stretches of about 20 to 30 amino acids and are well conserved in prokaryotes and eukaryotes. These sequences have a common structure, consisting of a positively charged n-region, followed by a hydrophobic h-region and a neutral, but polar, c-region. The signal peptide is cleaved by a specific protease while the polypeptide is being transported through the membrane. Secretory proteins often contain disulfide bonds which cannot be formed in the reducing environment of a cell-free lysate. The solubility and functionality of these proteins is therefore greatly reduced if they are expressed in a cell-free translation system.

Therefore, in another preferred embodiment of the method according to the invention the translated amino acid sequence is analyzed with respect to signal sequences and the corresponding cleavage sites. The SigP algorithm is preferably used for this purpose (H. Nielsen et al., Protein Eng. 1997, 10: 1-6). The prediction of signal peptide cleavage sites is very precise for Gram-negative bacterial sequences, whereas the prediction is generally less precise for eukaryotic and Gram-positive bacterial sequences, because their cleavage sites are not conserved to the same degree. On the basis of this prediction, information can be provided as to whether the sequence of the protein to be expressed already contains a signal peptide, which must be excised from the mature product at the specified position. In addition, experimental conditions can be suggested under which the problem of prevention of the formation of disulfide bonds can be circumvented.

The method according to the invention improves the expression efficiency of prokaryotic expression systems, particularly those which use the E. coli translation apparatus. The accuracy of the prediction is about 75%, but may be higher or lower in individual cases. The method according to the invention can be used to optimize the expression of proteins for scientific and/or commercial purposes, or to accelerate the expression of proteins or to make them cheaper.

The method according to the invention can of course also be used to predict the expression efficiency in eukaryotic expression systems, for which purpose specific attribute values of the expression constructs influencing expression efficiency in eukaryotic systems may have to be considered.

The example described below serves to illustrate the invention but should not in any way be understood in a restrictive sense.

### EXAMPLE

The following describes the procedure for setting up a predictive linkage, with which the expression efficiency of expression constructs based on the coding sequence can be calculated.

1. Data Base

1.1 Sequence Generation and Selection

Gene sequences from different organisms were selected for the performance of the expression experiments. The goal was to provide a representative set of prokaryotic and eukaryotic genes. For this purpose, about 200 human, 200 E. coli, 100 plant, 100 viral and 100 gene fragments from S. cerevisiae were provided, including the 39 bases starting with the second codon.

1.1.1 Sequence Sources

The Pedant databases of A. thaliana, E. coli and S. cerevisiae were used to extract the open reading frames. Open reading frames which have “hypothetical”, “putative”, “questionable”, “weak similarity”, “fragment”, “plasmid”, “patent” and “predicted” in the description text were not considered. In this way, 1341 A. thaliana, 1605 E. coli and 3909 S. cerevisae sequences were obtained. The human and viral sequences were retrieved from the EMBL database by using the following queries.
{EMBL}: [([Organism EQ text:homo;]) & ([Organism EQ text:sapiens;]) & (![AllText EQ text:hypothetical;]) & (![AllText EQ text:putative;])
& ([AllText EQ text:complete;]) & (![AllText EQ text:chromosome;]) & (![AllText EQ text:arm;]) &(![AllText EQ text:patent;])
& (![AllText EQ text:fragment;]) & (![AllText EQ text:putative;]) & (![AllText EQ text:cosmid;]) & (![AllText EQ text:“like”;])
& ([FtKey EQ text:“cds”;]) & (![AllText EQ text:“weak”;]) &(![AllText EQ text:“questionable”;]) & (![AllText EQ text:“partial”;])] or
({EMBL}: [([Organism EQ text:virus;]) & (![AllText EQ text:hypothetical;]) & (![AllText EQ text:putative;]) & ([AllText EQ text:complete;])
& (![AllText EQ text:chromosome;]) & (![AllText EQ text:arm;]) &(![AllText EQ text:patent;]) & (![AllText EQ text:fragment;])
& (![AllText EQ text:putative;]) & (![AllText EQ text:cosmid;]) & (![AllText EQ text:“like”;]) & ([FtKey EQ text:“cds”;])
& (![AllText EQ text:“weak”;]) &(![AllText EQ text:“questionable”;]) & (![AllText EQ text:“partial”;])])

9,162 human and 13,657 viral sequences were obtained in this way.

1.1.2 Selection Procedure

Specific gene sub-sequences were extracted for each organism which contained the 39 bases starting with the second codon. The sub-sequences were classified for each organism, using hierarchical clustering. Depending on the number of members of each class and the total number of desired sub-sequences, those sequences were extracted which were close to the class average.

As a result of this procedure, 221 human, 202 E. coli, 116 A. thaliana, 108 viral and 109 S. cerevisiae sequences were obtained. The gene sequence of the green fluorescence protein (GFP) and a hexa-his tag were added adjacent to the 3′ end of all sequences. A 2-stage PCR strategy was used to prepare all linear expression constructs. The 39 base pair sequences from the five different organisms were introduced through the primer of the first of these PCR reactions. The corresponding mRNA sequences were derived from the constructs prepared in this way and used as data base for the analysis described here. All constructs were expressed in a cell-free E. coli expression system (RTS Rapid Translation System RTS 100 E. coli HY Kit, Roche Diagnostics).

1.2 Initiation Region

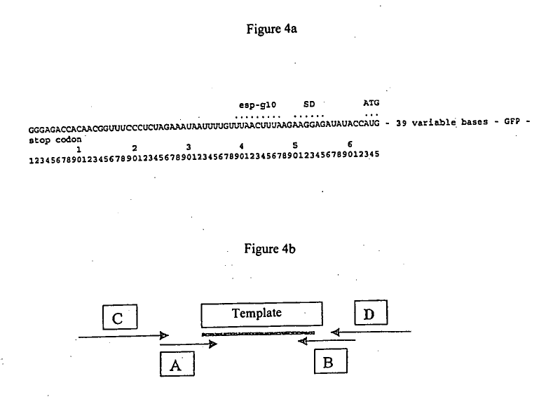

FIG. 4a illustrates the initiation region of all sequences; FIG. 4b illustrates the PCR strategy.

1.3 Expression Value Overview

The expression experiments were performed with the RTS 100 E. coli HY Kit Expression System from Roche Diagnostics GmbH. The expression of GFP was measured as internal control. All activity data were verified and compared with protein data (SDS-PAGE/Coomassie staining) and/or Western Blot analysis. All quantities expressed are given below as a relative percentage of the expression of GFP.

Three detection techniques were used: Fluorescence detection of the fusion protein GFP, densitometry of Coomassie-stained denaturing protein gels and Western Blot using antibody against the C-terminal His tag. The Coomassie value was used when no fluorescence was detectable, but a Western blot signal was present. In the other cases, the expression value was determined from the fluorescence signal.

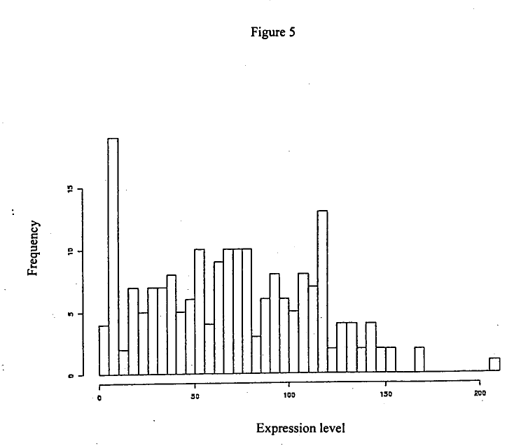

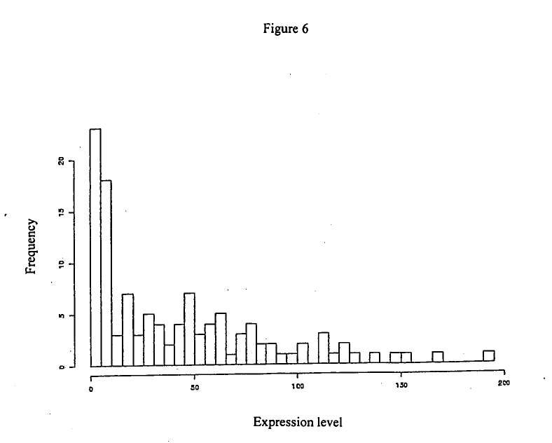

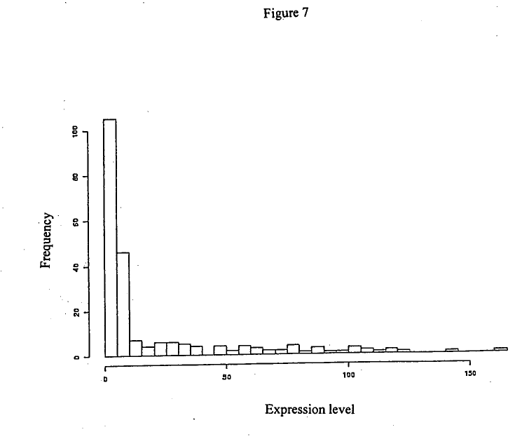

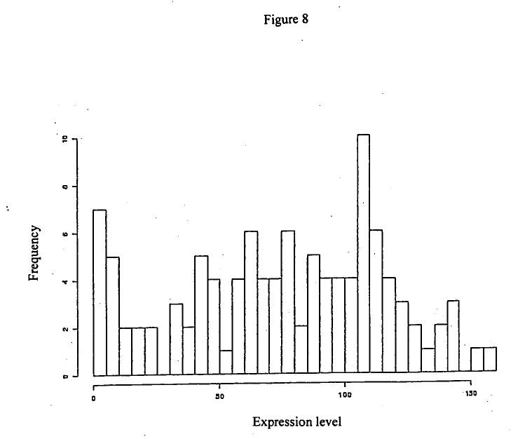

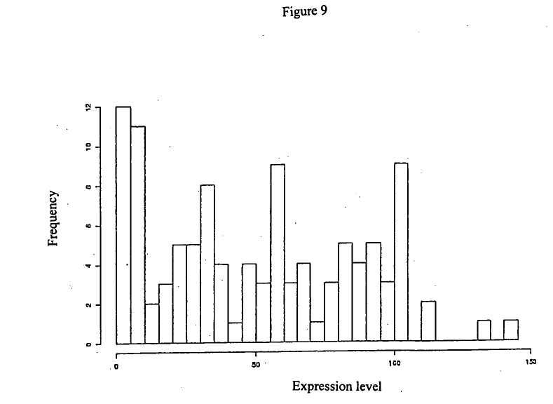

742 Sequences were included in the analysis (see Table 3 and FIG. 2). The relative expression levels were classified into so-called expression categories: high (exp>80), good (30<exp≦80), low (0<exp≦30) and none (exp=0). FIGS. 5 to 9 show expression histograms for all five sets of sequences.

TABLE 3
Expression data for the 742 Sequences

| | Mean | | | | | |
|---|---|---|---|---|---|---|
| Organism | Expression | High | Good | Low | None | Total |
| Homo | 20 | 16 | 29 | 79 | 95 | 219 |
| sapiens | | | | | | |
| E. coli | 71 | 79 | 79 | 44 | 0 | 202 |
| viral | 51 | 30 | 40 | 32 | 6 | 108 |
| A. thaliana | 36 | 13 | 33 | 40 | 18 | 104 |
| S. cerevisiae | 75 | 52 | 39 | 14 | 4 | 109 |
| Total | | 190 | 220 | 209 | 123 | 742 |

2. Sequence Attributes

The following attributes were determined on the basis of the DNA sequence.

2.1 Primary Structure
Length of the gene sequence
Length of the mRNA sequence

GC Content:

The content of G or C in the mRNA was calculated for various sequence stretches: gcX=GC content for base X; gc_cont_X_Y=average G/C content between bases X and Y (for example, gc_cont_{—}66_{—}85 means the fraction of G or C in bases 66 to 85 of the mRNA); gc_cont is the fraction of G or C in the entire mRNA.

Codon Adaptation Index (cai):

The codon adaptation index for E. coli according to Sharp and Li (1987) is calculated for various sequence stretches: caiX=cai of codon X; cai_X_Y=the cai of the sequence between codons X and Y; cai=the cai for the whole gene sequence.

Signal P Values:

Signal P is a program to identify the signal peptides of secretory proteins (http://www.cbs.dtu.dk/services/SignalP). Signal peptides have an average length of 26 amino acids. To accurately detect signal peptides by signal values, it is required to input an amino acid sequence of between 50 and 70 residues. Therefore, the first 70 amino acids of the original gene sequence, from which the 39 bases for the expression experiments were taken, were provided as input data for the determination of signal P values. If Signal P has detected a signal peptide, only the first 13 amino acids were actually present in the expression experiments. The results presented by Signal P include the values meanS_val and maxY_val, which indicate the presence of signal sequences.
Number of transmembrane helices of the translation products, determined by the ALOM2 algorithms.

The following abbreviations will be used below: pI for isoelectric point of the translation product, bX for base at position X of the gene sequence, coX for codon X of the gene sequence and aaX for amino acid X of the translated protein product.

2.2 Secondary Structure

The prediction of the secondary structure of the mRNA was performed with the software VIENNA RNA PACKAGE (Version 1.3, Ivo Hofacker, Department of Theoretical Chemistry, Währingerstr. 17, 1090 Vienna, Austria) with default energy parameters and an mRNA length of 300 bases. ppX is the binding probability of base X; ppwX is the energy-weighted binding probability of base X, correcting for the stability of the loop in which the base lies; ppweX is the energy-weighted binding probability of base X multiplied by the energy of the loop in which it lies.

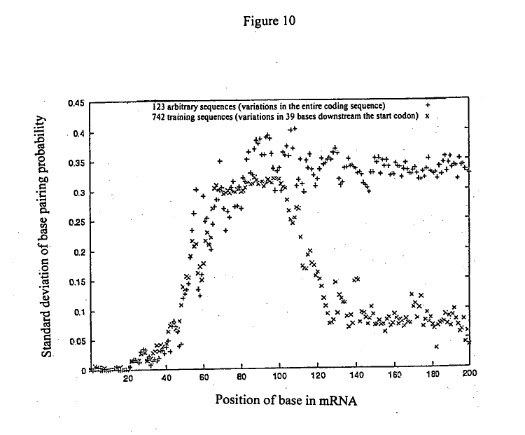

The standard deviations of the binding probabilities ppX in the data set are depicted as crosses in FIG. 10.

3. Identifying of Important Attributes

A preferred approach to find quantitative attributes which influence the measured expression level is to calculate the correlation values with the expression quantity. All 742 training data sets were included in the calculation of the training data sets. The correlation generally lay between −1 and +1, wherein a positive correlation means that the expression level rises with the attribute value, whereas a negative correlation means that the quantity expressed decreases while the attribute value rises.

3.1 Quantitative Primary Structure Attributes

Of all primary structure attributes, the GC content, particularly between bases 66 and 85, exhibited the most significant correlation with the expression levels (see Table 4).

TABLE 4
Primary structure attributes which exhibit
high correlations with the expression level

| Attribute | Correlation |
|---|---|
| gc_cont_41_80 | −0.55 |
| gc_cont_81_120 | −0.37 |
| gc_cont | −0.51 |
| gc_cont_66_85 | −0.56 |
| gc_cont_86_105 | −0.33 |
| maxY_val | −0.29 |
| meanS_val | −0.32 |
| gc66 | −0.31 |
| gc71 | −0.26 |
| gc77 | −0.27 |
| gc80 | −0.26 |

With other calculated quantitative primary structure attributes, such as the condon adaptation index, the correlation is in some cases less marked, but can also be of significance in individual cases.

3.2 Quantitative Secondary Structure Attributes

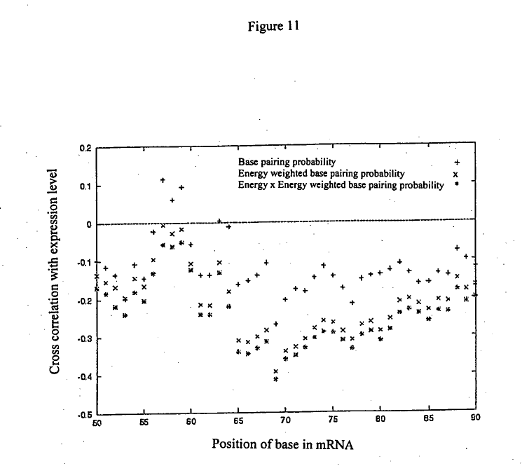

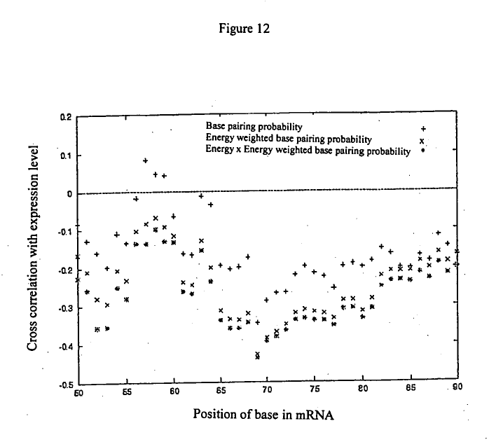

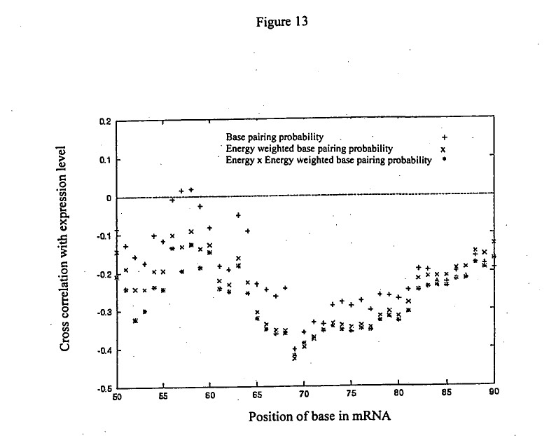

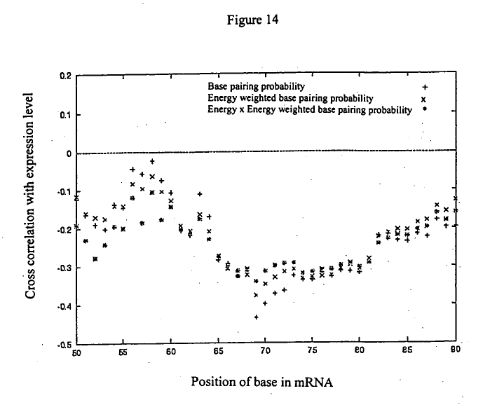

Table 5 lists high correlation values between the base pairing probabilities of specific bases and the expression level. The highest values are in a sequence region with a length of about 20 bases immediately adjacent to the start codon (see also FIGS. 11 to 14).

TABLE 5
Correlation values for secondary structure attributes
determined at T = 60° C.

| Base | Pp | ppw |
|---|---|---|
| 65 | −0.23 | −0.30 |
| 66 | −0.25 | −0.34 |
| 67 | −0.26 | −0.35 |
| 68 | −0.24 | −0.35 |
| 69 | −0.40 | −0.43 |
| 70 | −0.36 | −0.40 |
| 71 | −0.33 | −0.38 |
| 72 | −0.34 | −0.35 |
| 73 | −0.29 | −0.34 |
| 74 | −0.28 | −0.34 |
| 75 | −0.29 | −0.35 |
| 76 | −0.28 | −0.34 |
| 77 | −0.30 | −0.35 |
| 78 | −0.26 | −0.32 |
| 79 | −0.26 | −0.30 |
| 80 | −0.27 | −0.32 |

FIGS. 11 to 14 illustrate the three types of secondary structure attributes for bases 50 to 90 (there are no additional regions of high correlation in the range from base 1 to base 200). Four different temperatures were used for the secondary structure prediction. Base positions with high correlations are relatively insensitive to variations in prediction temperature for the attributes pp, ppw and ppwe. The correlation values of the three types differ more at lower temperatures and converge at higher temperatures. The energy weighted attributes, ppw and ppwe, generally give higher correlation values.

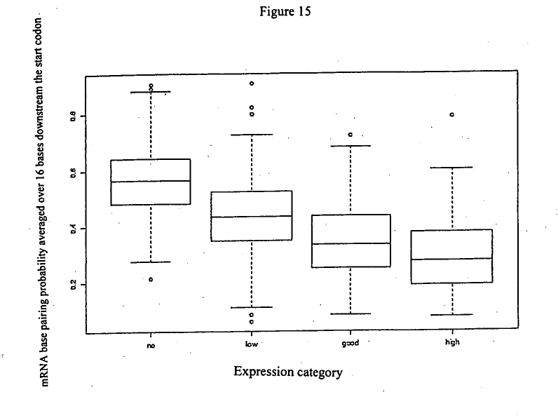

The pairing probabilities of base region 65 to 80 were averaged. FIG. 15 shows the box plot of this average ppav against the various expression categories. The correlation value of ppav and the quantity expressed is −0.537.

4. Regression Models

On the basis of the training data obtained as described above and with the help of regression models, a functional correlation was established between dependent and independent variables. In the present example, the quantitative sequence attributes were selected as independent variables, whereas the expression value is taken as the dependent variable. Only linear multivariate models were considered in the context of the present example, in other words the coefficients were fixed as linear. Non-linear variants are obviously also conceivable. A polynome of third order was used to improve the fit (see FIG. 3).

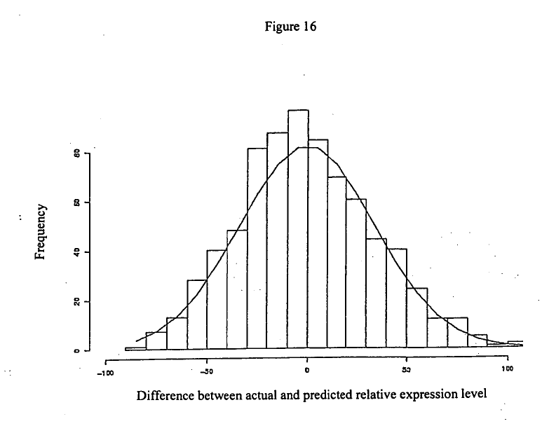

FIG. 16 illustrates the histograms of the differences between the predicted and actual expression values. This is a Gauss curve, centered at zero and with standard deviation of about 0.33 REE. About 68% of cases lie in the region of ±0.33 REE around the predicted value (the region under the Gauss curve between ±0.33 REE).

The accuracy is obtained from the sum of all correctly predicted cases divided by the sum of all predicted cases and comes to 0.79.

The accuracy of prediction was double-checked by repeating the fit, using only 80% of randomly selected cases in the training data. The predicted expression values of the remaining 20% of the data were then compared with the actual expression values. This analysis led to a Gauss curve which was about 0.40 REE in breadth (data not shown).

5. Decision Trees

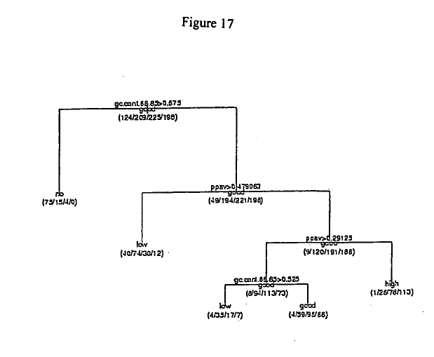

An alternative method of prediction employs machine-learning procedures, which establish a decision tree from a collection of cases belonging to known classes (J. R. Quinlan, C4.5: Programs for Machine Learning, Morgan Kaufmann, 1993). A classification based on the values of one of the attributes is performed at each node in the tree. A sequence of decisions must be made in order to reach one leaf in the tree. As defined above, the four categories of expression values form the classes “none”, “low”, “good” and “high”. FIG. 17 illustrates decision trees and Table 6 gives the corresponding values of the accuracy of prediction. For the derivation of the decision tree, it is adequate to the first approximation only to include the attributes ppav and gc_cont_{—}66_{—}85.

TABLE 6
Accuracy of prediction by the decision tree in FIG. 17.

| | Classifi- | Classifi- | Classifi- | Classifi- |
|---|---|---|---|---|
| | cation | cation | cation | cation |
| Expression | Probability | Probability | Probability | Probability |
| Class | none | low | good | high |
| none | 0.8 | 0.16 | 0.04 | 0 |
| low | 0.16 | 0.52 | 0.23 | 0.09 |
| good | 0.02 | 0.27 | 0.43 | 0.28 |
| high | 0.00 | 0.12 | 0.36 | 0.52 |

6. Discussion

With the help of the experiments described in this example, a data set has been provided which exhibits adequate sequence variability in the sequence region which is adjacent to the translation start codon, that is, in the 39 bases downstream of the translation start. The remaining DNA is completely constant for all 742 sequences.

A broad range of protein expression amount was observed in the data set—ranging from no expression to very high expression. A pool of several hundred attribute values was determined on the basis of the given sequences. Attributes were selected which correlate with or influence the amount of translation product. These are mainly the G/C content and the pairing probability in the mRNA secondary structure in the first 20 bases behind the translation start codon.

Regression models and decision trees were constructed on the basis of this subset of attributes. A prediction of the expected amount of translation product in a prokaryotic system can be made for a given sequence which originates from the same expression vector class used as in the training sequences. The accuracy of prediction is described as the probability that the expression amount lies in a given range. The probability is two thirds that the expression quantity lies within 40 expression units of the predicted expression (100 expression units is the expression quantity of GFP. On the basis of this distribution, other questions can be addressed which influence the success of expression in prokaryotic translation systems.

The accuracy of prediction as defined above can alternatively be described on the basis of the number of correctly and wrongly predicted test cases. The accuracy fluctuates between 65 and 85%, depending on the test data set.

1. A method for predicting the expression efficiency in the preparation of a protein by an expression system, comprising:
a) generating at least one expression construct comprising a sequence coding for the protein and flanking regulatory sequences;
b) determining at least one attribute value of the expression construct influencing the expression efficiency; and
c) calculating the expression efficiency of the expression construct by mutual linkage with at least one attribute value determined in step b).

2. A method according to claim 1, wherein the expression system is a prokaryotic system.

3. A method according to claim 2, wherein the prokaryotic expression system is a prokaryotic cell or an extract from the prokaryotic cell.

4. A method according to claim 3, wherein the prokaryotic cell is E. coli.

5. A method according to claim 1, wherein the attribute values of the expression construct which determine the expression efficiency are selected from the group consisting of quantitative primary structure attributes, qualitative primary structure attributes and quantitative secondary structure attributes.

6. A method according to claim 5, wherein the quantitative primary structure attributes comprise the G/C content in subregions or in the whole region of the expression construct.

7. A method according to claim 5, wherein the qualitative primary structure attributes comprise the first base of the second codon of the coding sequence and/or the base sequence of the second codon.

8. A method according to claim 5, wherein the quantitative secondary structure attributes comprise the base pairing probability for at least one of the bases in the mRNA sequence.

9. A method according to claim 5, wherein the quantitative secondary structure attributes comprise the base pairing probability for at least one of the bases in the mRNA sequence, in the region 100 bases upstream and 100 bases downstream of the start codon.

10. A method according to claim 5, wherein the quantitative secondary structure attributes comprise the base pairing probability for at least one of the bases in the mRNA sequence in the region 60 bases downstream and 60 bases upstream of the start codon.

11. A method according to claim 1, wherein the at least expression construct comprises a first expression construct comprising the native mRNA sequence coding for the protein and a second expression construct comprising a coding sequence which differs from a native mRNA sequence coding for the protein to be prepared by at least one base substitution.

12. A method according to claim 11, wherein the base substitution in the mRNA sequence coding for the protein leads to an identical amino acid or a conservative amino acid substitution in the protein.

13. A method according to claim 11, wherein the base substitution in the mRNA sequence coding for the protein leads to a substitution, insertion or deletion by one or more of the 20 naturally occurring amino acids in the protein.

14. A method according to claim 12 or 13, wherein the base substitution occurs within the first 30 codons of the translated region of the mRNA sequence coding for the protein.

15. A method according to claim 14, wherein the base substitution occurs within the first 15 codons of the translated region of the mRNA.

16. A method according to claim 14, wherein the base substitution occurs within the first seven codons of the translated region of the mRNA sequence coding for the protein.

17. A method according to claim 1, wherein the at least one expression construct comprises a first expression construct comprising the mRNA coding for the protein and a second expression construct comprising a coding sequence which differs from the native mRNA coding for the protein to be prepared by deletion of bases and/or insertion of bases.

18. A method according to claim 1, wherein the generation of the expression construct is performed upon consideration of at least one of a desired cloning strategy; incorporation of purification and/or detection tags; and number or type of permitted amino acid substitutions.

19. A method according to claim 1, wherein the calculation of the expression efficiency for the expression construct is performed by mutual linkage with at least one attribute value determined in b) by multiple regression of the dependence of experimentally determined expression yields on attribute values of the corresponding expression construct.

20. A method according to claim 19, wherein G/C content, base pairing probability or both are used as independent variables in the regression.

21. A method according to claim 1, wherein the calculation of the expression efficiency for the expression construct is performed by mutual linkage with at least one attribute value determined in b) by machine-learning methods which construct a decision tree of a set of cases belonging to known classes.

22. A method according to claim 1, wherein the calculation of the expression efficiency for the expression construct is performed by a Bayes network.

23. A method according to claim 1, further comprising analyzing physico-chemical properties of translation products derived from the expression construct.

24. A method according to claim 23, wherein the physico-chemical properties are selected from at least one of the group consisting of solubility, chaperone dependency and product fragmentation by proteolysis, false internal initiation of translation, premature termination of translation, and occurrence of secretory signal sequences.

25. A method according to claim 1, further comprising analyzing the expression construct for undesired fragmentation sites, and wherein expression constructs are generated in a) with which the fragmentation is minimized.

26. A method according to claim 25, wherein the undesired fragmentation sites occur within the coding sequence and comprise internal initiation sites, premature termination sites and/or rare codon clusters.

27. A method according to claim 25, wherein the undesired fragmentation sites occur within the protein product and comprise proteolytic cleavage sites.

28. A method according to claim 1, at least one of a)-c) is performed on a computer.

29. A method according to claim 28, wherein each of a)-c) is performed on a computer.

30. A method for predicting the expression efficiency in the preparation of a protein by an expression system, comprising:
a) providing a nucleic acid sequence which codes for the protein to be produced;
b) specifying constraints for incorporation of purification and/or detection tags;
and/or the number or type of permitted amino acid substitutions;
c) generating at least one expression construct containing a native sequence coding for the protein;
d) generating one or more modified expression constructs by nucleotide substitutions and/or insertions and/or deletions;
e) calculating the expression efficiency for each of the expression constructs in c) and d) by mutual linkage with at least one of the attribute values influencing the expression efficiency;
f) generating PCR primer sequences; and
g) outputting the expression efficiencies calculated for the expression constructs and/or the PCR primer sequences for the expression constructs.

31. A method for predicting the expression efficiency in the preparation of a protein by an expression system, comprising:
a) providing a nucleic acid sequence which codes for the protein to be prepared;
b) specifying constraints for the desired cloning strategy, the incorporation of purification and/or detection tags and/or the number or type of permitted amino acid substitutions;
c) selecting a suitable expression vector;
d) generating at least one expression construct containing a native sequence coding for the protein;
e) generating one or more modified expression constructs by nucleotide substitutions and/or insertions and/or deletions.
f) calculating the expression efficiency for each of the expression constructs in d) and e) by mutual linkage with at least one of the attribute values influencing the expression efficiency; and
g) outputting the expression efficiencies calculated for the expression construct(s).

32. A method according to claim 30 or 31, wherein data of the physico-chemical properties of the protein product and preferably suggestions for their improvement are provided.

33. A method for the preparation of a protein from an expression system, comprising:
a) predicting the expression efficiency according to the method of claim 1;
b) selecting an expression construct with a determined expression efficiency; and
c) producing the protein from the expression construct from b) in a cellular or cell-free expression system.

34. A method according to claim 33, wherein said selecting comprises selecting the expression construct having the highest expression efficiency.

35. A method according to claim 33, further comprising providing data for the physico-chemical properties of the protein.

36. A machine-readable medium, comprising instructions for performing a method for predicting the expression efficiency in the preparation of a protein in expression systems, preferably in prokaryotic expression systems, wherein the method comprises:
a) generating at least one expression construct comprising a sequence coding for the protein and flanking regulatory sequences;
b) determining at least one attribute value of the expression construct influencing the expression efficiency; and
c) calculating the expression efficiency of the expression construct by mutual linkage with at least one attribute value determined in b).

37. A medium according to claim 36, wherein said instructions are for performing the method on a computer.

38. Computer program product designed such that a method for predicting the expression efficiency in the preparation of a protein in expression systems, preferably in prokaryotic expression systems is performed when the computer program product is used on a computer, wherein the process comprises:
a) generating at least one expression construct comprising a sequence coding for the protein and flanking regulatory sequences;
b) determining at least one attribute value of the expression construct influencing the expression efficiency; and
c) calculating the expression efficiency of the expression construct by mutual linkage with at least one attribute value determined in b).
